(19) United States
(12) Patent Application Publication
(10) Pub. No.: US 2009/0247368 A1
(43) Pub. Date: Oct. 1, 2009

(54) SPORTS HEALTH CARE APPARATUS WITH PHYSIOLOGICAL MONITORING FUNCTION

(75) Inventor: Cheng-Tang Chiang, Taichung City (TW)

Correspondence Address: Muncy, Geissler, Olds & Lowe, PLLC, P.O. BOX 1364, FAIRFAX, VA, 22038-1364, US

(73) Assignee: BOSON TECHNOLOGY CO., LTD.

(21) Appl. No.: 12/059,735

(22) Filed: Mar. 31, 2008

(51) Int. Cl. A61B 5/00

(52) U.S. Cl. 482/8

(57) ABSTRACT

The present invention includes a handheld device and a main device, wherein the handheld device is held by an athlete and the main device is moved with the athlete. Through a measuring device acquiring a physiological condition of the athlete and a movement state of the handheld device and transmitting thereof to the main device, the main device can produce an exercise indication signal according thereto, so that the athlete can obtain an exercise direction and also can manage the physical condition during exercise.

### FIELD OF THE INVENTION

The present invention is related to a sports health care apparatus, and more particularly to a sports health care apparatus which can monitor real time physiological condition for providing message or alarm about physical fitness, so as to ensure the athlete a health body and prevent athletic damage.

### BACKGROUND OF THE INVENTION

Currently, many assistant devices are developed for monitoring physiological condition of athlete, such as U.S. Pat. No. 6,945,940. The device acquires the physiological condition of athlete, for example, heartbeat, for showing the athlete the heartbeat variation thereof, so as to avoid sports intensity from exceeding the bearable level of heat and lung function. However, this device is incapable of monitoring the impact loading on the bones and the joints, so that the problem that the bones and joints might have injury during exercise still can not be solved.

### SUMMARY OF THE INVENTION

Therefore, the object of the present invention is to disclose a sports health care apparatus for monitoring the impact born by the athlete, so as to avoid bones and joints from injury.

For achieving the object described above, the present invention includes a handheld device and a main device. The handheld device is held by an athlete and has a measuring device, wherein the measuring device has a measuring module and a first acceleration module for acquiring a physiological condition of the athlete and a movement state of the handheld device, so as to produce a signal of measuring device. The main device is moved with the athlete and receives the signal of measuring device, wherein the main device includes a second acceleration module for detecting the movement state of the main device and a multimedia output media, and according to the signal of measuring device and the movement state of the main device, the main device produces an exercise indication signal, which is outputted by the multimedia output media for providing the athlete an exercise direction.

Accordingly, when the athlete's body suffers an excess impact loading, through the main device moving with the athlete and the second acceleration module detecting the impact, the main device can produce the exercise indication signal to warn the athlete to stop exercise or lower down the intensity.

### BRIEF DESCRIPTION OF THE DRAWINGS

The foregoing aspects and many of the attendant advantages of this invention will be more readily appreciated as the same becomes better understood by reference to the following detailed description, when taken in conjunction with the accompanying drawings, wherein:

FIGS. 1A˜1B are block diagrams for showing a measuring device and a main device according to the present invention;

FIG. 2 is a schematic view showing the structure of the present invention;

FIG. 3 is a schematic view showing the present invention as applying to running;

FIG. 4 is a schematic view showing the present invention as applying to a sports game machine; and

FIG. 5 is a block diagram showing the transmission with external device in the present invention.

### DESCRIPTION OF THE PREFERRED EMBODIMENT

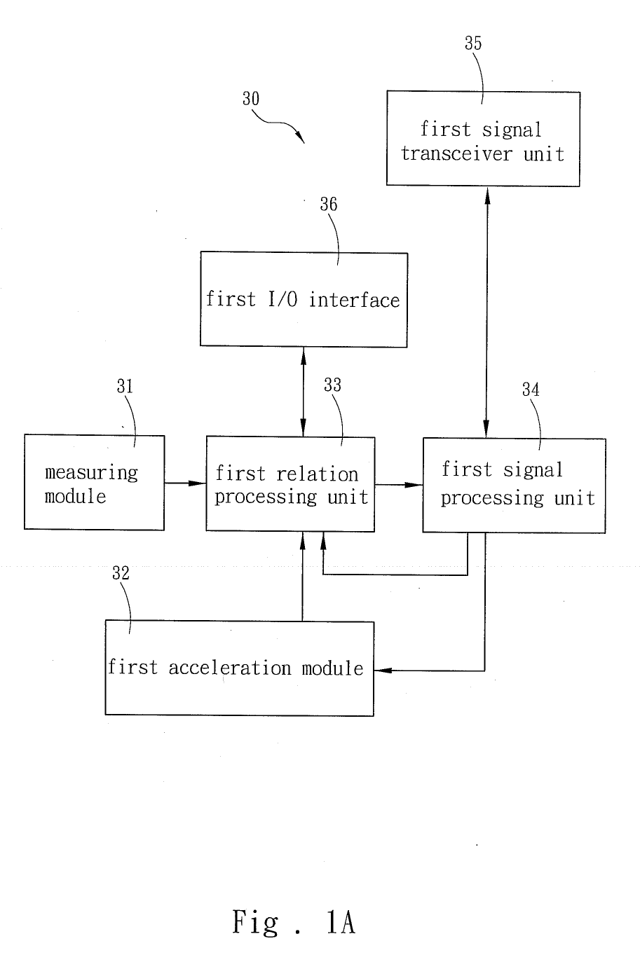

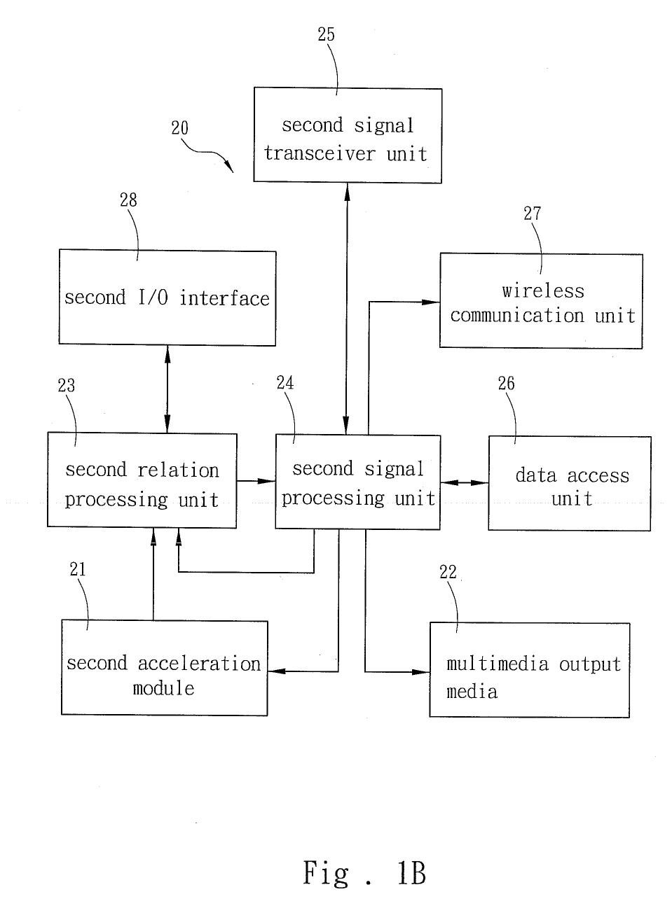

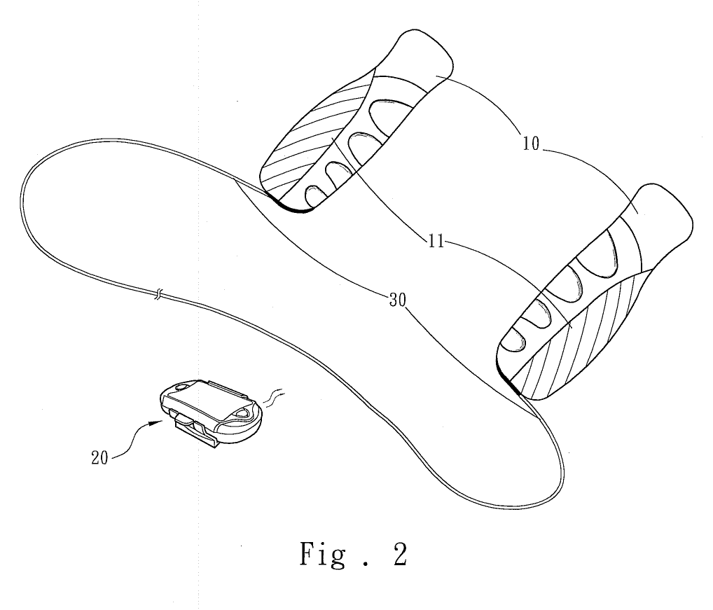

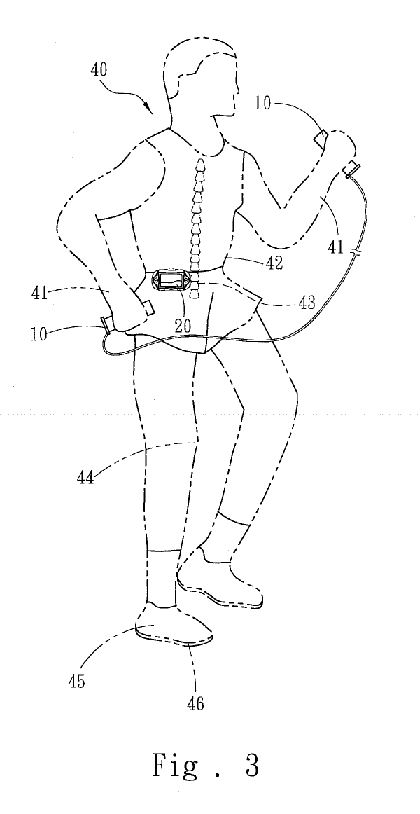

Please refer to FIG. 1A, FIG. 1B and FIG. 2. The present invention is related to a sports health care apparatus capable of monitoring physiological condition which is provided to an athlete 40 (as shown in FIG. 3). The apparatus includes a handheld device 10 and a main device 20, wherein the handheld device 10 is provided to be held by the athlete 40, and the handheld device 10 has a measuring device 30 mounted thereon, which includes a measuring module 31, a first acceleration module 32, a first relation processing unit 33, a first signal processing unit 34 and a first signal transceiver unit 35. The measuring module 31 measures the physiological condition of the athlete 40 by acquiring the physiological potential through contacting the sensing pieces 11 on the handheld device 10 with the athlete 40. The first acceleration module 32 measures the movement state of the handheld device 10 held by the athlete 40. The first relation processing unit 33 receives signals from the first signal processing unit 34, the first acceleration module 32 and the measuring module 31. After the first signal processing unit 34 processes the signals, a signal of measuring device is produced and transmitted by the first signal transceiver unit 35 to the main device 20.

The main device 20 is set to be moved with the athlete 40 and to receive the signal of measuring device. The main device 20 includes a second acceleration module 21 for detecting the movement state of the main device 20, a multimedia output media 22, a second relation processing unit 23, a second signal processing unit 24 and a second signal transceiver unit 25. The first signal transceiver 35 and the second signal transceiver unit 25 are connected with each other, so that the main device 20 can receive the signal of measuring device. The second relation processing unit 23 receives signals from the second signal processing unit 24 and the second acceleration module 21, and after the second signal processing unit 24 processes the signals, an exercise indication signal is produced and outputted by the multimedia output media 22, so that the user can have the exercise in accordance with the exercise indication signal. The multimedia output media 22 can be a display screen or an audio output device, which can be identified by the athlete 40.

The present invention further includes a first I/O interface 36 connected with the first relation processing unit 33, a data access unit 26 connected with the second signal processing unit 24, a wireless communication unit 27 connected with the second signal processing unit 24, and a second I/O interface 28 connected to the second relation processing unit 23, wherein the wireless communication unit 27 is a muddle to long distant wireless communication element selected from WIFI and WIMAX, so as to provide a remote transmission or monitoring function. Moreover, the first signal transceiver unit 35 and the second signal transceiver 25 can be connected to a short distant wireless communication element selected from Bluetooth, infrared ray, Zigbee and 2.4 GHz frequency hopping, for facilitating transmission. The first I/O interface 36, the data access unit 26 and the second I/O interface 28 are provided for the athlete 40 to achieve personal settings, such as personal data and control parameters, so as to meet personal demands.

Please refer to FIG. 3. As shown, the present invention can be applied to the running athlete 40. The athlete 40 holds the handheld device 10 by hands 41, so that the sensing pieces 11 and the measuring device 30 can acquire the physiological condition of the athlete 40 and also the movement state of the handheld device 10. The main device 20 is hanged on the athlete's body 42, and thus, the second acceleration module 21 in the main device 20 can detect the reacting force produced from the floor which passes through the shoes 45, the knees 44 and the spine 43 and reaches the main device 20. Therefore, after setting the maximum tolerant impact and accumulated impact according to personal physiological condition, if the reacting force exceeds the tolerant range, the multimedia output media 22 can output the exercise indication signal to warn the athlete 40, so that the athlete 40 can slow down or stop the exercise, exchange for proper sports shoes 45, or adjust the cushion coefficient of the cushion pad 46, thereby protecting the knees 44 and the spine 43 of the athlete 40 from injury.

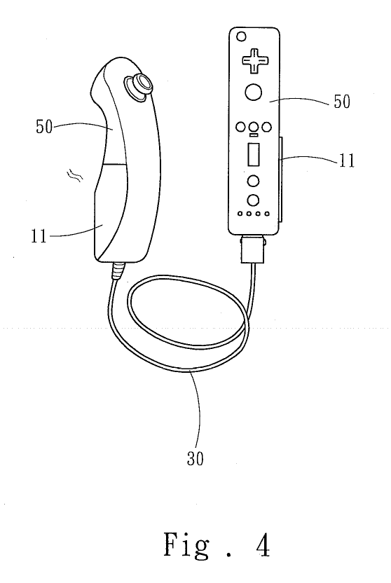

Please refer to FIG. 4. The present invention also can be applied to an emulated sports game. Here, the sensing pieces 11 of the handheld 10 are implemented to be mounted on the joystick 50 of a game machine (not shown). Thereby, when the athlete 40 holds the joystick 50 to play the game, the sensing pieces 11 and the measuring device 30 can acquire the physiological condition of the athlete 40 and also the movement state of the joystick 50, and then transmit thereof to the main device 20 or the game machine.

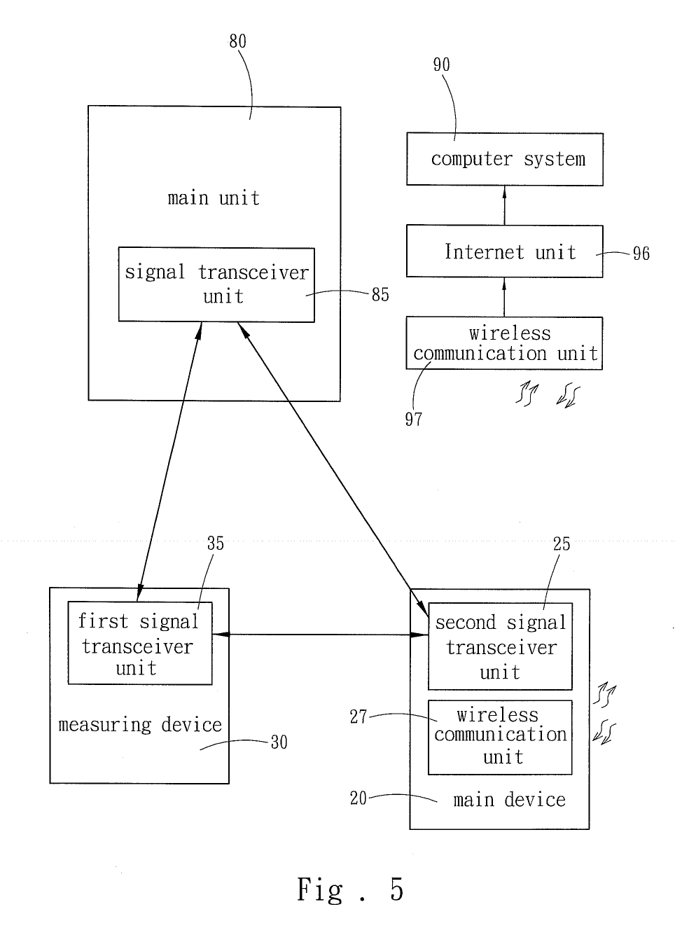

Please refer to FIG. 5. The main device 20 and the measuring device 30 can be connected with a signal transceiver unit 85 in the main unit 80 respectively through the second signal transceiver unit 25 and the first signal transceiver unit 35, so as to achieve a real time interaction. Furthermore, the main device 20 can be connected with a computer system 90 through the wireless communication unit 27, another wireless communication unit 97 and an Internet unit 96, so as to achieve a remote transmission or remote monitoring.

In the aforesaid, the present invention detects the physiological condition and impact intensity for preventing bones and joints from suffering excess impact and thus from injury, so that the present invention can be applied to many other fields, such as sports, game, aerobic exercise and rehabilitation. In addition, the present invention also utilizes the first acceleration module 32 and the second acceleration module 21 to acquire the movement state, so as to decide the type of exercise and provide a proper monitoring and exercise indication signal.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A sports health care apparatus with physiological monitoring function for an athlete, comprising:
a handheld device, held by the athlete, having a measuring device, wherein the measuring device has a measuring module and a first acceleration module for acquiring a physiological condition of the athlete and a movement state of the handheld device, so as to produce a signal of measuring device; and
a main device, moved with the athlete and receiving the signal of measuring device, wherein the main device comprises a second acceleration module for detecting the movement state of the main device and a multimedia output media, and according to the signal of measuring device and the movement state of the main device, the main device produces an exercise indication signal to be outputted by the multimedia output media so as to provide the athlete an exercise direction.

2. The apparatus as claimed in claim 1, wherein the measuring device further comprises a first relation processing unit, a first signal processing unit and a first signal transceiver unit, wherein the measuring module measures the physiological condition of the athlete, the first acceleration module measures the movement state of the handheld device held by the athlete, the first relation processing unit receives signals from the first signal processing unit, the first acceleration module and the measuring module, and after the first signal processing unit processes the signals, a signal of measuring device is produced and transmitted by the first signal transceiver unit to the main device.

3. The apparatus as claimed in claim 2, further comprising a first I/O interface connected with the first relation processing unit.

4. The apparatus as claimed in claim 2, wherein the main device further comprises a second relation processing unit, a second signal processing unit and a second signal transceiver unit, wherein the first signal transceiver and the second signal transceiver are connected with each other, so that the main device receives the signal of measuring device, the second relation processing unit receives signals from the second signal processing unit and the second acceleration module, and after the second signal processing unit processes the signals, an exercise indication signal is produced.

5. The apparatus as claimed in claim 4, further comprising a data access unit connected with the second signal processing unit.

6. The apparatus as claimed in claim 4, further comprising a wireless communication unit connected with the second signal processing unit.

7. The apparatus as claimed in claim 4, further comprising a second I/O interface connected to the second relation processing unit.

8. The apparatus as claimed in claim 1, wherein the main device is hanged on the body of the athlete.

9. The apparatus as claimed in claim 8, wherein the second acceleration module in the main device detects a reacting force produced from the floor which passes through the shoes, the knees and the spine and then reaches the main device, and the multimedia output media outputs the exercise indication signal to warn the athlete.
